(19) United States
(12) Patent Application Publication
(10) Pub. No.: US 2023/0257704 A1
(43) Pub. Date: Aug. 17, 2023

(54) METHODS FOR PREPARATION OF IMMUNE CELLS

(71) Applicant: Cellular Biomedicine Group Inc., Rockville, MD (US)

(72) Inventors: Fei WANG, Shanghai (CN); Dan ZHANG, Shanghai (CN); Li ZHANG, Shanghai (CN); Jiaqiang REN, Shanghai (CN); Guozhi CHEN, Shanghai (CN); Weide LIN, Shanghai (CN); Junfeng WU, Shanghai (CN); Luyi ZHANG, Shanghai (CN)

(21) Appl. No.: 18/001,306

(22) PCT Filed: Jun. 11, 2021
(86) PCT No.: PCT/US2021/037055

§ 371 Date: Dec. 9, 2022

(30) Foreign Application Priority Data
Jun. 12, 2020 (CN) 2020105366876

(51) Int. Cl. C12N 5/0783; C07K 16/28; C12N 15/86; C12M 1/26

(52) U.S. Cl. CPC C12N 5/0636; C07K 16/2809; C07K 16/2818; C12N 15/86; C12M 33/10; C12N 2740/16043

(57) ABSTRACT

Methods for preparation of immune cells in a fully closed system are provided. Specifically, the methods contain the steps of pretreating, cell sorting, activation, transduction and expansion. The present methods greatly improve the preparation efficiency of immune cells, and reduce preparation costs.

### CROSS REFERENCE TO RELATED APPLICATION

The present application claims priority to Chinese patent application No. 2020105366876, filed Jun. 12, 2020, which is hereby incorporated by reference in its entirety.

### TECHNICAL FIELD

The present disclosure relates to the fields of biotechnology, and more specifically to methods for the automatic preparation of immune cells, e.g., in a fully closed system.

### BACKGROUND

In cellular immunotherapy, also known as adoptive cell therapy, a patient's immune cells are collected for genetic modification or selective expansion to enhance the antigen-specific immune responses. In recent years, tumor immunotherapy has performed well in clinical practice. However, the preparation methods of immune cells are relatively complicated. Insufficiency of any components of the process, such as process flow, equipment and facilities, and reagents, can have an important impact on the quality of the final cell preparation, which in turn affects clinical effects. Therefore, a fully automatic and fully closed preparation process can ensure product safety and batch-to-batch consistency to the maximum extent, reduce the impact of personnel and the environment, as well as improve the production efficiency of immune cell therapy products.

The traditional immune cell preparation cycle is about 10 to 14 days. This relatively long time period not only is likely to cause excessive differentiation and senescence of the cells during the culture process, but also can greatly affect the disease progression of the patients. Thus, more rapid preparation process is more in line with clinical needs. It can improve clinical efficacy, reduce costs and increase the production capacity. To meet the characteristics and needs of each cell type, the culture system may contain serum, be low serum, or be free of serum.

Therefore, there is an urgent need to develop more robust and efficient methods of generating immune cells for clinical use.

### SUMMARY

The present disclosure provides for a method for culturing genetically modified immune cells. The method may comprise: (a) providing a sample containing immune cells; (b) optionally, washing the sample to obtain pretreated immune cells; (c) sorting the pretreated immune cells to obtain enriched immune cells; (d) activating the enriched immune cells with microbeads coated with activating agents to obtain activated immune cells; (e) genetically modifying the activated immune cells to obtain genetically modified immune cells; and (f) optionally, expanding the genetically modified immune cells.

The microbeads may have a diameter ranging from about 1 μm to about 10 μm, from about 2 μm to about 8 μm, or from about 4 μm to about 5 μm.

In step (d), the activating may be performed with a microbead-to-cell ratio ranging from about 0.1 to about 10, from about 0.2 to about 8, from about 0.5 to about 8, from about 0.1 to about 8, from about 0.5 to about 5, from about 0.5 to about 4, from about 0.5 to about 3, from about 0.5 to about 2, from about 0.5 to about 1, from about 1 to about 8, from about 1 to about 6, from about 1 to about 5, from about 1 to about 3, from about 0.5 to about 5, from about 1 to about 2, about 0.1, about 0.2, about 0.5, about 0.8, about 1, about 1.2, about 1.5, about 1.8, about 2, about 2.5, about 3, about 3.5, about 4, about 4.5, or about 5.

The activating agents may be antibodies or fragments thereof, cytokines, recombinant costimulatory molecules, small drug inhibitors, or combinations thereof. In certain embodiments, the activating agents are anti-CD3 and/or anti-CD28 antibodies or fragments thereof.

In step (d), the activating may be performed for about 2 hours to about 1 week, about 2 hours to about 6 days, about 2 hours to about 5 days, about 2 hours to about 4 days, about 2 hours to about 3 days, about 2 hours to about 2 days, about 2 hours to about 1 day, about 2 hours to about 20 hours, about 2 hours to about 16 hours, about 4 hours to about 5 days, about 4 hours to about 96 hours, about 4 hours to about 48 hours, about 4 hours to about 36 hours, about 4 hours to about 24 hours, about 4 hours to about 20 hours, about 4 hours to about 16 hours, about 16 hours to about 48 hours, about 16 hours to about 40 hours, about 16 hours to about 36 hours, about 16 hours to about 24 hours, about 2 hours, about 4 hours, about 5 hours, about 6 hours, about 8 hours, about 10 hours, about 12 hours, about 18 hours, about 20 hours, about 24 hours, about 30 hours, about 36 hours, about 40 hours, about 48 hours, about 50 hours, about 55 hours, about 60 hours, about 65 hours, about 72 hours, about 84 hours, about 96 hours, about 4.5 days, about 5 days, about 5.5 days, about 6 days, about 6.5 days, or about 1 week.

Step (d), step (e) and step (f) of the method, or all steps of the method, may be performed in about 2 days to about 5 days, about 3 days to about 4 days, about 2 days to about 10 days, about 2 days to about 9 days, about 2 days to about 8 days, about 2 days to about 7 days, about 2 days to about 6 days, about 2 days to about 5 days, about 2 days to about 4 days, about 3 days to about 10 days, about 3 days to about 9 days, about 3 days to about 8 days, about 3 days to about 7 days, about 3 days to about 6 days, about 3 days to about 5 days, about 2 days, about 3 days, about 4 days, about 5 days, about 6 days, about 7 days, about 8 days, about 9 days, or about 10 days.

Steps (c)-(f) of the method, steps (d)-(f) of the method, or all steps of the method, may be performed in a closed and sterile system.

The immune cells may be T cells, T cell subsets, or natural killer (NK) cells.

In step (d), the activating may be performed with an immune cell density ranging from about 0.5×10⁶ cells/ml to about 10×10⁶ cells/ml, from about 2×10⁶ cells/ml to about 3×10⁶ cells/ml, from about 0.5×10⁶ cells/ml to about 8×10⁶ cells/ml, from about 0.5×10⁶ cells/ml to about 5×10⁶ cells/ml, from about 1×10⁶ cells/ml to about 8×10⁶ cells/ml, from about 1×10⁶ cells/ml to about 6×10⁶ cells/ml, from about 1×10⁶ cells/ml to about 5×10⁶ cells/ml, from about 1×10⁶ cells/ml to about 4×10⁶ cells/ml, from about 1×10⁶ cells/ml to about 3×10⁶ cells/ml, about 0.5×10⁶ cells/ml, about 1×10⁶ cells/ml, about 1.5×10⁶ cells/ml, about 2×10⁶ cells/ml, about 2.5×10⁶ cells/ml, about 3×10⁶ cells/ml, about 3.5×10⁶ cells/ml, about 4×10⁶ cells/ml, about 4.5×10⁶ cells/ml, about 5×10⁶ cells/ml, about 5.5×10⁶ cells/ml, about 6×10⁶ cells/ml, about 6.5×10⁶ cells/ml, about 7×10⁶ cells/ml, about 7.5×10⁶ cells/ml, about 8×10⁶ cells/ml, about 8.5×10⁶ cells/ml, about 9×10⁶ cells/ml, about 9.5×10⁶ cells/ml, or about 10×10⁶ cells/ml.

In step (e), the genetically modifying may be transducing or transfecting.

In step (e), the genetically modifying may comprise introducing into the activated immune cells a polynucleotide encoding a chimeric antigen receptor (CAR) or a T cell receptor (TCR).

In step (e), the genetically modifying may comprise transducing the activated immune cells with lentiviral vectors, gamma-retroviral vectors, alpha-retroviral vectors, or adenoviral vectors. In certain embodiments, the genetically modifying may comprise transducing the activated immune cells with lentiviral vectors.

In step (b), the washing may comprise using a human serum albumin (HSA) solution having a HAS final concentration of about 0.1% to about 30%, about 0.1% to about 10%, about 0.1% to about 25%, about 0.1% to about 20%, about 0.1% to about 15%, about 0.1% to about 8%, about 0.1% to about 6%, or about 0.1% to about 5%.

In step (b), the washing may comprise centrifuging the sample using a centrifugal force ranging from about 100×g to about 1,000×g, from about 200×g to about 400×g, from about 100×g to about 800×g, from about 100×g to about 600×g, from about 100×g to about 500×g, from about 200×g to about 800×g, from about 200×g to about 600×g, or from about 200×g to about 500×g.

In step (b), the washing may comprise centrifuging the sample for about 100 seconds to about 600 seconds, about 300 seconds to about 400 seconds, about 50 seconds to about 1,000 seconds, about 100 seconds to about 800 seconds, about 100 seconds to about 500 seconds, about 200 seconds to about 800 seconds, about 200 seconds to about 600 seconds, about 200 seconds to about 500 seconds, about 300 seconds to about 800 seconds, or about 300 seconds to about 500 seconds.

In step (b), the washing may comprise diluting the sample about 0 to about 5 folds, about 1 to about 5 folds, about 1 to about 4 folds, about 1 to about 3 folds, or about 2 to about 3 folds.

In step (b), the washing may comprise performing/repeating the washing cycle for 1 to 5 times, 1 to 4 times, 1 to 3 times, 1 to 2 times, 2 to 5 times, 2 to 4 times, or 2 to 3 times.

Step (b) may have an output volume ranging from about 5 ml to about 400 ml, from about 20 ml to about 100 ml, from about 10 ml to about 300 ml, from about 10 ml to about 200 ml, from about 10 ml to about 100 ml, from about 20 ml to about 400 ml, from about 20 ml to about 300 ml, from about 20 ml to about 200 ml, from about 50 ml to about 400 ml, from about 50 ml to about 300 ml, from about 50 ml to about 200 ml, or from about 50 ml to about 100 ml.

In step (c), the sorting may comprise using anti-CD4 and/or anti-CD8 antibodies or fragments thereof.

The sample may be peripheral blood, immune cells, monocyte collections, or peripheral blood mononuclear cells (PBMCs), e.g., from a subject (e.g., patient), or a plurality of subjects.

The present disclosure provides genetically modified immune cells prepared by the present method.

The present disclosure also provides a cell preparation comprising the genetically modified immune cells.

Also encompassed by the present disclosure is a pharmaceutical composition comprising the genetically modified immune cells. The pharmaceutical composition may further comprise a pharmaceutically acceptable carrier.

An object is to provide a method for fully closed automatic preparation of an immune cell, e.g., in a fully closed system.

In a first aspect, a method for culturing an immune cell is provided, wherein the method comprises the following steps:

(a) providing a sample containing immune cells,

(b) optionally, performing washing pretreatment on the sample containing immune cells to obtain pre-treated immune cells,

(c) performing cell sorting on the pretreated immune cells to obtain sorted immune cells,

(d) activating the sorted immune cells to obtain activated immune cells, wherein the activation treatment is performed by using an activator selected from TransAct™, Dynabeads® and OKT3 antibodies,

(e) transducing/transfecting the activated immune cells to obtain transduced/transfected immune cells, and

(f) culturing the transduced/transfected immune cells to prepare desired immune cells.

In another embodiment, the steps (c), (d), (e) and (f) are performed on Prodigy® equipment.

In another embodiment, in step (d), the activator is Dynabeads®, wherein preferably the amount of Dynabeads® is Dynabeads: cells=0.1 to 10, more preferably Dynabeads: cells=0.5 to 5.

In another embodiment, in step (d), the activator is TransAct™, wherein preferably the amount of TransAct™ is 0.1 to 30 ml, more preferably 1 to 10 ml.

In another embodiment, in step (d), the activator is an OKT3 antibody, wherein preferably the amount of the OKT3 antibody is 10 ng/ml to 50 mg/ml (final concentration).

In another embodiment, in step (d), the density of immune cells subjected to activation treatment is (0.1-20)×10⁶ cells/ml, preferably (0.5-10)×10⁶ cells/ml.

In another embodiment, in step (d), the time for activation treatment is 4 to 96 h, preferably 16 to 48 h.

In another embodiment, in step (d), the number of activated cells is 1×10⁵ to 20×10⁹.

In another embodiment, the immune cells include T cells and natural killer (NK) cells.

In another embodiment, the sample containing immune cells is selected from peripheral blood, immune cells, monocyte collections, and PBMC.

In another embodiment, the sample includes a fresh sample and a frozen sample.

In another embodiment, the amount of the sample is 5 to 500 ml, preferably 10 to 100 ml.

In another embodiment, in step (b), the washing pretreatment is performed on equipment selected from Sepax 2, Sepax C-pro, Sefia, Lovo, CS 5+, CS Elite and a centrifuge.

In another embodiment, in step (b), the centrifugal force of the washing pretreatment is 100 to 1,000 g, preferably 200 to 400 g.

In another embodiment, in step (b), the centrifugal time of the washing pretreatment includes but is not limited to 100 to 600 seconds, preferably 300 to 400 seconds.

In another embodiment, in step (b), the dilution ratio of the sample for the washing pretreatment is 0 to 5 folds, preferably 1 to 3 folds;

In another embodiment, in step (b), the number of washing cycles of the washing pretreatment is 1 to 5, preferably 1 to 3; In another embodiment, in step (b), the output volume of the sample after washing includes but is not limited to 5 to 400 ml, preferably 20 to 100 ml.

In another embodiment, in step (c), the amount of immune cells for cell sorting is 1×10⁵ to 50×10⁹ cells, preferably (1−10)×10⁹ cells.

In another embodiment, in step (c), the sorting comprises positive sorting and/or negative sorting.

In another embodiment, the marker of the positive sorting is selected from CD4⁺, CD8⁺, CD62L⁺, CD3⁺, CD56⁺, or a combination thereof.

In another embodiment, the marker of the negative sorting marker is selected from CD14⁺, CD19⁺, CD269⁺, or a combination thereof.

In another embodiment, the sorting is performed by using an anti-CD4 antibody and/or an anti-CD8 antibody, or fragments thereof. For example, the anti-CD4 antibody reagent is a CliniMACS CD4 reagent diluted 3 to 5 times, and the anti-CD8 antibody reagent is a CliniMACS CD8 reagent diluted 3 to 5 times. In another preferred embodiment, the dilution is performed by using PBS-EDTA containing 0.1% to 10% HSA, preferably, the dilution is performed by using PBS-EDTA containing 0.2% to 1% HSA, and more preferably, the dilution is performed by using PBS-EDTA containing 0.4% to 0.6% HSA.

In another embodiment, in step (c), the volume of the anti-CD4 antibody reagent and the anti-CD8 antibody reagent used is 1.5 to 10 ml, preferably 4 to 6 ml.

In another embodiment, in step (c), the solvent of a sorting reagent containing the anti-CD4 antibody reagent and the anti-CD8 antibody reagent is PBS-EDTA containing 0.1% to 10% HSA.

In another embodiment, the ratio of the anti-CD4 antibody reagent, the anti-CD8 antibody reagent and immune cells is 100×10⁶ to 1,000×10⁶ cells/mL.

In another embodiment, the method for diluting the anti-CD4 antibody reagent and the anti-CD8 antibody reagent is as follows: dividing 7.5 ml (a whole bottle) of a CliniMACS CD4 reagent or a CliniMACS CD8 reagent into 1.5 ml/bottle, and then adding 3.5 ml PBS-EDTA containing 0.1% to 10% HSA to prepare into a total of 5 ml of a diluted reagent for sorting.

In another embodiment, in step (e), the transduction/transfection treatment is selected from non-viral transfection (including an electroporation system, such as a Neon transfection system and a Maxcyte transfection system), and viral transduction (including a lentivirus system, an adenovirus system and an adeno-associated virus vector).

In another embodiment, in step (e), a virus is used for transduction, and the multiplicity of infection or MOI is 0 to 1,000, preferably 1 to 10.

In another embodiment, the virus titer is 1×10⁵ to 10×10¹⁰ TU/ml.

In another embodiment, in step (f), a culture medium used in the culture comprises: a serum medium, a low serum medium, or a serum-free medium.

In another preferred embodiment, in step (f), the seeding cell density of the culture is 0.01×10⁶/ml to 20×10⁶/ml.

In another embodiment, in step (f), the time for cell culture is 4 to 96 h, preferably 16 to 48 h.

In another embodiment, after step (f), the method further comprises the following steps: (g) washing and concentrating cultured immune cells to obtain concentrated immune cells; and/or (h) dispensing the (concentrated) immune cells.

In another embodiment, the washing and concentrating is performed on equipment selected from Sepax 2, Sepax C-pro, Sefia, Lovo, CS 5⁺, CS Elite and a centrifuge.

In another embodiment, the volume of the sample to be washed and concentrated is to 500 ml.

In another embodiment, in step (g), the centrifugal force for washing the sample is 100×g to 1,000×g.

In another embodiment, in step (g), the centrifugation time for washing the sample is 100 to 600 seconds.

In another embodiment, in step (g), the dilution ratio of the sample is 0 to 5 folds.

In another embodiment, in step (g), the number of washing cycles is 1 to 5.

In another embodiment, in step (g), the output volume after concentration is 5 to 400 ml.

In another embodiment, in step (h), the dispensing is performed on equipment selected from Sepax 2, Sepax C-pro, Sefia and Prodigy®.

In another embodiment, in step (h), the volume of the sample to be dispensed is 5 ml to 500 ml.

In another embodiment, in step (h), the output volume of the dispensing is 5 ml to 400 ml.

In another embodiment, the proportion of naïve T (Tnaive) cells in the sorted/enriched immune cells obtained in step (c) is more than 20%, more than 25%, more than 30%, more than 35%, more than 40%, more than 45%, or more than 50%.

In another embodiment, the sorting recovery rate in step (c) is more than 30%, more than 35%, more than 40%, more than 45%, more than 50%, more than 55%, more than 60%, more than 65%, more than 70%, more than 75%, more than 80%, more than 85%, or more than 90%.

In another embodiment, the positive rate of the activated immune cells obtained in step (d) is more than 30%, more than 35%, more than 40%, more than 45%, more than 50%, more than 55%, more than 60%, more than 65%, more than 70%, more than 75%, more than 80%, more than 85%, or more than 90%.

In another embodiment, step (d), step (e) and step (f), or all steps of the method, are completed in 2 to 12 days, in 2 to 10 days, in 2 to 8 days, in 2 to 6 days, in 2 to 5 days, in 3 to 12 days, in 3 to 10 days, in 3 to 8 days, in 3 to 6 days, in 3 to 4 days, in 2 to 5 days, in 3 to 5 days, in 2 days, in 3 days, in 4 days, in 5 days, in 6 days, in 7 days, in 8 days, in 9 days, or in 10 days.

In another embodiment, the ratio (V1/V2) of the volume V1 of immune cells obtained in step (f) to the volume V2 of immune cells before the activation treatment in step (d) is 6 to 30, preferably 8 to 12.

In another embodiment, the proportion of Tnaive cells in the immune cells obtained in step (f) is more than 20%, more than 25%, more than 30%, more than 35%, more than 40%, more than 45%, more than 50%, more than 55%, or more than 60%.

In another embodiment, the ratio (T1/T2) of the amount of Tnaive cells, T1, in the immune cells obtained in step (f) to the amount of Tnaive cells, T2, in the immune cells obtained in step (d) is (0.8 to 1.2):(0.8 to 1.2). The ratio (T3/T4) of the amount of central memory T (Tcm) cells, T3, in the immune cells obtained in step (f) to the amount of Tcm cells, T4, in the immune cells obtained in step (d) is (0.8 to 1.2):(0.8 to 1.2).

In a second aspect, cultured immune cells are provided, where the immune cells are prepared by the present method.

In a third aspect, a cell preparation is provided, where the cell preparation contains (a) the present immune cells, and (b) a pharmaceutically acceptable carrier.

In another embodiment, the cell preparation is a liquid preparation (such as an injection).

It should be understood that within the scope of the present invention, the above-mentioned technical features of the present invention and the technical features specifically described in the following text (such as the embodiments) can be combined with each other to form a new or preferred technical solution. Due to limited space, it will not be repeated one by one here.

### BRIEF DESCRIPTION OF THE DRAWINGS

FIG. 1 shows the amount and positive rate of cells sorted by Prodigy®.

FIG. 2 shows a cell subset after activation with Dynabeads®.

FIG. 3 shows a cell subset after activation with TransAct™.

FIG. 4 shows the amount of cells and the positive rate during the culture process after activation by different activating agents (or activators).

### DETAILED DESCRIPTION

The present method is robust and leads to equal or higher amounts of immune cells (e.g., genetically modified T cells) suitable for clinical application, e.g., compared to the standard CliniMACS Prodigy® automated processes. The present method can provide better transduction efficiency and robust manufacturing of clinically relevant numbers of genetically modified T cells.

The clinical manufacture of genetically modified T cells is currently a complex process that generally starts with obtaining the patient's peripheral blood mononuclear cells (PBMC). Current protocols feature a leukapheresis step. PBMC are often enriched for T cells and activated prior to gene modification with viral or nonviral vectors. The modified T cells are then expanded in order to reach the cell numbers required for treatment, after which the cells are finally formulated and/or cryopreserved prior to reinfusion into the patient. The cell product is subjected to a number of quality control assays and has to meet release criteria and Good Manufacturing Practices (GMP) guidelines.

The present disclosure provides for a method for culturing genetically modified immune cells. The method may comprise: (a) providing a sample containing immune cells; (b) optionally, washing the sample to obtain pretreated immune cells; (c) sorting the pretreated immune cells to obtain enriched immune cells; (d) activating the enriched immune cells with microbeads coated with activating agents to obtain activated immune cells; (e) genetically modifying the activated immune cells to obtain genetically modified immune cells; and (f) optionally, expanding the genetically modified immune cells.

In certain embodiments, the method is performed through an automated process in a device suitable for cell processing in a closed GMP-compliant environment (a closed and sterile cell culture system).

Cell processing in a closed GMP-compliant system may be performed e.g., with the CliniMACS Prodigy® and associated tubing sets (Miltenyi Biotec GmbH, Germany). The CliniMACS Prodigy® offers a flexible platform for cell processing applications enabling the magnetic separation of different cell types as well as cell processing protocols.

CliniMACS Prodigy® is an equipment that can provide a fully closed integrated production of a variety of cell products. The culture mode is different from the general manual preparation of immune cells. The automatic mechanical control reduces the time of manual operation. The fully closed system reduces the risk of contamination, and the standardized preparation process reduces the error of manual operation and improves production efficiency and quality of immune cell therapy products. See U.S. Pat. Nos. 10,131,876; 10,119,970; 10,620,212; 10,705,090; 10,705,091; 8,727,132; 8,747,290; 9,625,463; and 9,714,945, relating to cell preparation systems such as CliniMACS Prodigy®.

The present method may comprise automated cell preparation, selection (separation) of immune cells, activation of the cells, expansion of the cells, transduction of the cells, and formulation (wash) of the cells, e.g., for subsequent clinical use.

In certain embodiments, all steps are performed in a single device, e.g., the CliniMACS Prodigy®, using a single use closed and sterile tubing set and programmed software. The present methods can lead to high transduction efficiency of the manufactured T cells and a high transgene expression by the genetically modified immune cells.

Moreover, a large number of highly viable immune cells can be generated robustly in less than 5 days.

The immune cells may be enriched through magnetic separation using antigen-binding molecules (e.g., antibodies or fragments thereof) specific for a cell surface marker on the surface of the immune cells, such as markers CD2, CD3, CD4, CD8 CD25, CD28, CD27, CD45RA, CD45RO, CD62L, CD95, CD127, CD137, alpha/beta TCR, gamma/delta TCR, CCR7, PD-1 or Lag3.

The activating agents may be agonistic antibodies, cytokines, recombinant costimulatory molecules, small drug inhibitors, or combinations thereof. In certain embodiments, the activating agents are anti-CD3 and/or anti-CD28 antibodies or fragments thereof, coupled to microbeads, microparticles, microsphere or microstructures. In certain embodiments, the activating agents are microbeads coated with anti-CD3 and/or anti-CD28 antibodies or fragments thereof.

In certain embodiments, the microbeads are polymer microbeads. In certain embodiments, the microbeads are magnetic microbeads. In certain embodiments, the microbeads are magnetic polymer microbeads. In certain embodiments, the microbeads are superparamagnetic polymer microbeads. Polymers may include polystyrene, polyesters, polyethers, polyacrylates, polyacrylamides, polyamines, polyethylene imines, polyquarternium polymers, polyphosphazenes, polyvinylalcohols, polyvinylacetates, polyvinylpyrrolidones, block copolymers, or polyurethanes.

The microbeads may have a diameter (or a median diameter) ranging from about 1 μm to about 50 μm, from about 1 μm to about 40 μm, from about 1 μm to about 30 μm, from about 1 μm to about 20 μm, from about 1 μm to about 15 μm, from about 1 μm to about 12 μm, from about 1 μm to about 10 μm, from about 1 μm to about 8 μm, from about 1 μm to about 6 μm, from about 1 μm to about 5 μm, from about 2 μm to about 10 μm, from about 2 μm to about 8 μm, from about 2 μm to about 6 μm, from about 3 μm to about 10 μm, from about 3 μm to about 8 μm, from about 3 μm to about 6 μm, from about 3 μm to about 5 μm, from about 4 μm to about 5 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, or about 4.5 μm.

The genetic modification of immune cells may be performed by transduction, transfection or electroporation.

Transduction may be performed with lentiviruses, gamma-retroviruses, alpha-retroviruses or adenoviruses. Electroporation or transfection of the cells may be performed by introducing into the cells nucleic acids (DNA, mRNA, miRNA, antagomirs, ODNs), proteins, through site-specific nucleases (zinc finger nucleases, TALENs, CRISP/R), self-replicating RNA viruses (e.g. equine encephalopathy virus) or integration-deficient lentiviral vectors.

In one embodiment, the genetic modification of immune cells may be by transducing the cells with lentiviral vectors.

Expansion of the genetically modified immune cells may be performed by adding a suited cell medium for cell culture expansion, such as TexMACS GMP Medium (Miltenyi Biotec GmbH) to the cultivation chamber.

In certain embodiments, activation, genetic modification and/or expansion of immune cells, may be performed under shaking or rotating conditions. In one embodiment, the shaking is performed during expansion of the immune cells. The shaking (rotating) in the cultivation chamber may take place sporadically or periodically by rotating the cultivation chamber (centrifugation chamber) every 1-120 seconds, every 15-60 seconds, or every 30 seconds. The centrifugal forces may be between larger (>) 0 and maximum 70×g (1 to 1000 rpm in a chamber having a radius of 6 cm) in one or two directions, between 0.2 and 17×g (50 to 500 rpm in a chamber having a radius of 6 cm) in one or two directions, at 6×g (300 rpm in a chamber having a radius of 6 cm) in two directions. Importantly, the shaking conditions can be adapted during the culture (for example, increased with increased cell density) to best support the immune cell expansion.

Activation of the cells may be performed by using cell densities between 0.2×10⁶ cells per ml to 4×10⁶ cells per ml, between 0.5×10⁶ cells per ml to 2×10⁶ cells per ml, or about 1×10⁶ cells per ml. Alternatively, the activation may be performed by using high cell densities between 4×10⁶ cells per ml to 2×10⁷ cells per ml, or between 4×10⁶ cells per ml to 1×10⁷ cells per ml.

The genetically modified immune cells may be genetically modified to express a chimeric antigen receptor (CAR), a T cell receptor (TCR), or any accessory molecule, on their cell surface.

For final formulation, the expanded and genetically modified immune cells may be washed by centrifugation and replacement of culture medium with a buffer appropriate for subsequent applications (e.g., infusion of the generated cell composition into a patient).

When required, genetically modified immune cells can be separated from non-modified immune cells, e.g., using again the magnetic separation.

In another aspect, the present method provides a substantially pure composition of genetically modified immune cells. In certain embodiments, the present method generates about 80% to about 100%, at least or about 50%, at least or about 55%, at least or about 60%, at least or about 65%, at least or about 70%, at least or about 75%, at least or about 80%, at least or about 85%, at least or about 90%, at least or about 91%, at least or about 92%, at least or about 93%, at least or about 94%, at least or about 95%, at least or about 96%, at least or about 97%, at least or about 98%, at least or about 99%, or about 100% of the desired immune cells (e.g., genetically modified immune cells) in the cell composition.

In a further aspect, the present disclosure provides a pharmaceutical composition comprising the genetically modified immune cells.

In one embodiment, the CliniMACS Prodigy® and associated tubing sets (Miltenyi Biotec GmbH, Germany) are used herein as a closed cell sample processing system on which an automated process was implemented. But it is not intended to restrict the use of the present method to the CliniMACS Prodigy® system.

The CliniMACS Prodigy® System is designed to automate and standardize complete cellular product manufacturing processes. It combines CliniMACS® Separation Technology (Miltenyi Biotec GmbH, Germany) with a wide range of sensor-controlled, cell processing capabilities. Features of the device include: disposable CentriCult™ Chamber enabling standardized cell processing and cultivation; cell enrichment and depletion capabilities, alone or combined with CliniMACS® Reagents (Miltenyi Biotec GmbH); cell cultivation and cell expansion capabilities; final product formulation in pre-defined medium and volume; the possibility to program the device using Flexible Programming Suite (FPS) and GAMP5 compatible programming language for customization of cell processing; and tailor-made tubing sets for a variety of applications.

The step of sorting/separation of immune cells may comprise one, several (two or more) or a combination of positive enrichment steps, i.e., separation of T cells, T cell subsets and/or T cell progenitors (direct magnetic labeling). T cells may be selected for CD4+ and/or CD8+ T cells by using antigen binding molecules coupled to particles such as magnetic beads specific for CD4 and/or CD8. A subpopulation of T cells such as naïve and central memory T cells may be separated e.g., by using the marker CD62L.

The step of sorting/separation of immune cells may also comprise negative enrichment (direct labeling of non-T cells) of T cells or of the depletion of cellular subsets to be removed from the preparation. For example, B cells may be removed via the CD19 marker. Inhibitory cells such as regulatory T cells (CD25 high), monocyte (CD14) can be removed as well using the markers CD25 and CD14, respectively.

Viral transduction of the immune cells can be enhanced by the use of transduction enhancer reagents, especially transduction enhancer reagents including, but not limited to, polycationic reagents (polybrene, protamine sulphate, poly-L-lysine, peptides with a net positive charge), poloxamers, adhesion molecules such as fibronectin or modified fibronectin (RetroNectin), or protein targeting domains such as antibodies, antibody complexes, magnetic particles. The transduction enhancers can be provided in solution, coated on the cultivation chamber or coated on a carrier substance present in suspension/solution within the cultivation chamber.

In one embodiment, the centrifugation chamber and the cultivation chamber may be identical. The centrifugation chamber and the cultivation chamber can be used in various conditions: for example, for separation or transduction, high rotational speed (i.e., high g-forces) can be applied, whereas for example, culturing steps may be performed with slow rotation or even at idle state. In one embodiment, the chamber changes direction of rotation in an oscillating manner that results in a shaking of the chamber and maintenance of the cell in suspension. Accordingly, T cell activation, gene modifying and/or cultivation steps can be performed under steady or shaking conditions of the centrifugation or the cultivation chamber.

In one embodiment, a patient sample, for example, comprising immune cells of interest are introduced into the chamber of a closed and sterile culture system such as the CliniMACS Prodigy®. The sample is centrifugated, preferentially using optical density phase detection, excess erythrocytes are removed, the cell sample is washed using, e.g., the CliniMACS Buffer (Miltenyi Biotec GmbH) to avoid cell aggregation, and magnetically labeled with a magnetic cell separation reagent such as CliniMACS CD4 and CD8 Reagent (Miltenyi Biotec GmbH). After labeling, cells are washed, magnetically enriched via an integrated magnetic cell selection column and then returned to a cell culture chamber.

In the cell culture chamber, the immune cells may be activated upon steady or shaking culture conditions with one or a combination of activating agents capable of inducing immune cell (e.g., T cell) proliferation, such as agonistic antibodies (e.g. anti-CD3 and anti-CD28), cytokines (e.g. IL-1b, IL-2, IL-4, IL-6, IL-7, IL-9, IL-10, IL-12, IL-15, IL-17, IL-21, IL-22, IL-23, IL-35, TGF-b, IFN alpha, IFN gamma, TNF alpha) recombinant proteins, costimulatory molecules, lectins, ionophores, synthetic molecules, antigen presenting cells (APCs), artificial APCs, feeders, and combinations thereof. These activation reagents can be provided in solution, coated on the cultivation chamber or coated on a carrier substance present in suspension/solution within the cultivation chamber or on large particles.

Immune cells can be cultivated upon steady or shaking culture conditions. In certain embodiments, after a period of culture, viral vectors are added to the culture chamber and the cells are transduced. Following a further cell culture period, the cells can be transduced again or washed extensively and harvested (formulated). Prior to in vivo transfer of the genetically modified immune cell products, the cells can be washed, concentrated and resuspended in a buffer compliant with clinical requirements for in vivo infusion. Two or more (e.g., all) steps mentioned above may be performed automatically.

In one embodiment, immune cells are labeled by binding to antibody-coupled magnetic beads to a cell surface marker present on the surface of the immune cells, and enriching the labeled cells by magnetic separation (positive enrichment).

In another embodiment, the immune cells are enriched by binding to antibody-coupled magnetic beads to a cell surface marker not present on the surface of the T cells or defined cellular subsets and depleting the labeled cells by magnetic separation (negative enrichment).

In a further embodiment, in addition to the first enrichment of immune cells, the genetically modified immune cells are enriched in a second enrichment step by magnetic labeling of the genetically modified immune cells, and magnetic separation before or after cultivation to obtain higher frequency of the genetically modified immune cells in the finally achieved cell composition obtained by the present method. For example, if the genetically modified cell is a T cell expressing a CAR or TCR, the second separation step may be performed by using an antigen-binding molecule coupled to a magnetic particle specific for the recombinantly expressed CAR or TCR on the cell surface of the genetically modified T cell.

In one embodiment, a sample, e.g., whole blood from a patient, comprising immune cells, is provided. The sample may be connected to a closed and sterile cell culture system, e.g., the sample is connected via tubing sets to the CliniMACS Prodigy® device. The cell sample may be prepared by centrifugation in a centrifugation chamber of the device, resulting in the separation of erythrocytes and platelets from other cells including immune cells. Magnetic separation of immune cells may be performed by using antibodies coupled to magnetic particles specific for markers of immune cells, such as CD2, CD3, CD4, CD8 CD25, CD28, CD27, CD45RA, CD45RO, CD62L, CD95, CD127, CD137, alpha/beta TCR, gamma/delta TCR, CCR7, PD-1 or Lag3. Passing the labeled cells through a magnet unit with separation column of the device results in an enrichment of the immune cells. After moving the separated immune cells to the cultivation chamber (which may be identical to the centrifugation chamber) of the device, the cells are set at a given density of 0.5×10⁶ cells per ml to 2×10⁶ cells per ml to be activated by using activating agents described herein. The activated immune cells are then genetically modified in the cultivation chamber of the device, e.g., they are transduced with a lentiviral vector comprising a polynucleotide sequence encoding a CAR. Then the genetically modified immune cells are expanded in the cultivation under shaking conditions. Shaking may be performed by sporadic or periodical centrifugation of the cultivation chamber (in this case the cultivation chamber is identical to the centrifugation chamber) under conditions which allow the cells to be in suspension (and as disclosed herein). Finally, the cultured cells are washed by centrifugation, thereby allowing the replacement of culture medium with a buffer appropriate for subsequent applications such as infusion of the generated cell composition to a patient.

In one embodiment of the invention, a higher purity of transduced immune cells, e.g., T cells expressing a transgene such as a CAR or TCR on their cell surface, is obtained at the end of the manufacturing process. An additional cell selection step may be used to specifically enrich the genetically modified immune cells. For example, magnetic particles coated with antibodies directed against the surface molecule encoded by the transgene may be used for the selection step. The step of enrichment may be carried out by using again the magnetic separation unit of the device in an automated manner and is done before final formulation.

In certain embodiments, selection agent that can be removed from the surface of the selected cells after this second enrichment and before application to a patient or downstream use is used.

In another embodiment, it is possible to start the automated manufacturing process with higher cell densities by activating the immune cells under suspension conditions. When sufficient numbers of target immune cells can be obtained from the starting material, it is possible to start the automated manufacturing process with a high cell density of 4e6 to 1e7 immune cells directly under shaking conditions, under conditions which allow the cells to be in suspension for activation of the cells upon onset of the culture Immune cells can be further modified using lentiviral vector and expanded under suspension. In this embodiment, the shaking conditions may be maintained during the activation, genetic modification and expansion steps of the process as disclosed herein to keep the high-density cell culture in suspension.

In one embodiment, the immune cells may be genetically modified using lentiviral vectors. In one embodiment, lentiviral vectors with the VSVG pseudotype enable efficient transduction under automated manufacturing method. Other types of lentiviral vectors may also be used, such as measles virus (ML-LV), gibbon ape leukaemia virus (GALV), feline endogenous retrovirus (RD114), baboon endogenous retrovirus (BaEV) derived pseudotyped envelopes). Other viral vectors such as gamma or alpha retroviral vectors can be used. Transduction enhancer reagents may be added.

The terms “closed cell sample processing system” and “closed and sterile (cell culture) system” may be used interchangeably.

The term “closed and sterile (cell culture) system” as used herein refers to any closed system which reduces the risk of cell culture contamination while performing culturing processes such as the introduction of new material, e.g., by transduction, and performing cell culturing steps such as proliferation, differentiation, activation, and/or separation of cells. Such a system allows to operate under GMP or GMP-like conditions (“sterile”) resulting in cell compositions which are clinically applicable. For example, the CliniMACS Prodigy® (Miltenyi Biotec GmbH, Germany) may be used as a closed cell sample processing system. This system is disclosed in WO2009/072003. But it is not intended to restrict the present method to the CliniMACS Prodigy®.

The present method may be performed in a closed and sterile system (a closed cell sample processing system), comprising a centrifugation chamber comprising a base plate and cover plate connected by a cylinder, pumps, valves, a magnetic cell separation column and a tubing set. The blood samples or other sources comprising T cells, T cell subpopulations and/or T cell progenitors may be transferred to and from the tubing set by sterile docking or sterile welding.

The closed cell sample processing system may comprise a plurality of tubing sets where cells are transferred between the tubing sets by sterile docking or sterile welding.

The terms “automated method” or “automated process” as used herein refer to any process being automated through the use of devices and/or computers and computer software. Methods (processes) that have been automated require less human intervention and less human time. In some instances, the present method is automated if at least one step of the present method is performed without any human support or intervention. Preferentially the automated process is implemented on a closed cell sample processing system such as CliniMACS Prodigy® as disclosed herein.

The closed cell sample processing system may comprise a) a sample processing unit comprising an input port and an output port coupled to a rotating container (or centrifugation chamber) having at least one sample chamber, wherein the sample processing unit is configured to provide a first processing step to a sample or to rotate the container so as to apply a centrifugal force to a sample deposited in the chamber and separate at least a first component and a second component of the deposited sample; and b) a sample separation unit coupled to the output port of the sample processing unit, the sample separation unit comprising a separation column holder, a pump, and a plurality of valves configured to at least partially control fluid flow through a fluid circuitry and a separation column positioned in the holder, where the separation column is configured to separate labeled and unlabeled components of sample flown through the column.

The rotating container may also be used as a temperature controlled cell incubation and cultivation chamber (CentriCult Unit=CCU). This chamber may be flooded with defined gas mixes, provided by an attached gas mix unit (e.g., use of pressurized air/N2/CO2 or N2/CO2/O2).

All agents may be connected to the closed system before process initiation. This comprises all buffers, solutions, cultivation media and supplements, microbeads, used for washing, transferring, suspending, cultivating, harvesting cells or immunomagnetic cell sorting within the closed system. Alternatively, such agents might by welded or connected by sterile means at any time during the process.

The sample may be provided in transfer bags or other suited containers which can be connected to the closed system by sterile means.

The sample may be a human cell sample of hematologic origin. For example, the cell sample may be composed of hematologic cells from a donor or a patient. Such blood product can be in the form of whole blood, buffy coat, leukapheresis, PBMCs or any clinical sampling of blood product. It may be from fresh or frozen origin.

The centrifugal step may comprise one, more or all of the following aspects: gradient separation, erythrocyte reduction, platelet removal and cell washing.

In certain embodiments, washing means the replacement of the medium or buffer in which the cells are kept. The replacement of the supernatant can be in part or entirely. Several washing steps may be combined in order to obtain a more complete replacement of the original medium in which the cells are kept. A washing step may involve pelleting the cells by centrifugation forces and removing the supernatant. Cells may be pelleted by rotation of the chamber at, e.g., 300×g, and the supernatant is removed during rotation of the chamber. Medium is added during rotation or at a steady state.

The term “shaking condition” may refer to any means that can keep the cells in suspension. The shaking may be performed by rotating (or sporadic centrifugation) a cultivation chamber of a closed and sterile cell culture system, where the rotation is performed continuously or periodically. The shaking may also be performed e.g., by using a whipping equipment, a propelling device or a flow of liquid (e.g., channels) integrated into the closed and sterile cell culture system used which prevent sedimentation of the cells.

The term “marker” may refer to a cell antigen that is specifically expressed by a certain cell type. Preferentially, the marker is a cell surface marker so that enrichment, isolation and/or detection of living cells can be performed. The markers may be positive selection markers such as CD4, CD8 and/or CD62L, or may be negative selection markers (e.g., depletion of cells expressing CD14, CD16, CD19, CD25, CD56).

The term “antigen-binding molecule” as used herein refers to any molecule that binds preferably to or is specific for the desired target molecule of the cell, i.e., the antigen. The term “antigen-binding molecule” comprises e.g., an antibody or antibody fragment. The term “antibody” as used herein refers to polyclonal or monoclonal antibodies. The antibody may be of any species, e.g., murine, rat, sheep, human, etc. For therapeutic purposes, if non-human antigen binding fragments are to be used, these can be humanized by any method known in the art. The antibodies may also be modified antibodies (e.g., oligomers, reduced, oxidized and labeled antibodies).

The term “antibody” comprises both intact molecules and antibody fragments, such as Fab, Fab′, F(ab′)2, Fv and single-chain antibodies. Additionally, the term “antigen-binding molecule” includes any molecule other than antibodies or antibody fragments that binds preferentially to the desired target molecule of the cell. Suitable molecules include, without limitation, oligonucleotides known as aptamers that bind to desired target molecules, carbohydrates, lectins or any other antigen binding protein (e.g., receptor-ligand interaction). The linkage (coupling) between antibody (or antibody fragment) and microbeads can be covalent or non-covalent. A covalent linkage may be, e.g., the linkage to carboxyl-groups on polystyrene beads, or to NH2 or SH2 groups on modified beads. A non-covalent linkage may be, e.g., via biotin-avidin or a fluorophore-coupled-particle linked to anti-fluorophore antibody.

A potent sorting technology is magnetic cell sorting. Methods to separate cells magnetically are commercially available e.g., from Invitrogen, Stem cell Technologies, in Cellpro, Seattle or Advanced Magnetics, Boston. For example, monoclonal antibodies can be directly coupled to magnetic polystyrene particles like Dynabeads® or similar magnetic particles and used, e.g., for cell separation. The cells are isolated by placing the tube on a magnetic rack. These microbeads can be either directly conjugated to monoclonal antibodies or used in combination with anti-immunoglobulin, avidin or anti-hapten-specific microbeads. Cells can be separated by incubating them with magnetic microbeads coated with antibodies directed against one or more particular surface antigens. This causes the cells expressing this antigen to attach to the magnetic microbeads. With this method, the cells can be separated positively or negatively with respect to the particular antigen(s). The procedure can be performed using direct magnetic labeling or indirect magnetic labeling. For direct labeling, the specific antibody is directly coupled to the magnetic microbeads. Indirect labeling is a convenient alternative when direct magnetic labeling is not possible or not desired. A primary antibody, a specific monoclonal or polyclonal antibody, a combination of primary antibodies, directed against any cell surface marker can be used for this labeling strategy. The primary antibody can either be unconjugated, biotinylated, or fluorophore-conjugated. The magnetic labeling is then achieved with anti-immunoglobulin microbeads, anti-biotin microbeads, or anti-fluorophore microbeads. The above-described processes can also be performed in a closed cell sample processing system such as CliniMACS® (Miltenyi Biotec GmbH, Germany) or CliniMACS Prodigy® (Miltenyi Biotec GmbH, Germany).

The term “genetically modified cell” means cells containing and/or expressing a foreign gene or nucleic acid sequence which in turn modifies the genotype or phenotype of the cell or its progeny. Especially, the term refers to the fact that cells can be manipulated by recombinant methods well known in the art to express stably or transiently peptides or proteins, e.g., CARs, which are not expressed in these cells in the natural state. Genetic modification of cells may include but is not restricted to transfection, electroporation, nucleofection, transduction using retroviral vectors, lentiviral vectors, non-integrating retro- or lentiviral vectors, transposons, designer nucleases including zinc finger nucleases, TALENs or CRISPR/Cas.

The genetically modified immune cells, obtainable by the methods disclosed herein may be used for subsequent steps such as research, diagnostics, pharmacological or clinical applications known to the person skilled in the art.

The genetically modified immune cells can also be used as a pharmaceutical composition in the therapy, e.g., cellular therapy, or prevention of diseases. The pharmaceutical composition may be transplanted into an animal or human, for example a human patient. The pharmaceutical composition can be used for the treatment and/or prevention of diseases in mammals, especially humans, possibly including administration of a pharmaceutically effective amount of the pharmaceutical composition to the mammal. Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The composition of genetically modified immune cells, obtained by the present method may be administered either alone, or as a pharmaceutical composition in combination with diluents and/or with other components such as cytokines or cell populations. Briefly, pharmaceutical compositions of the present invention may comprise the genetically modified immune cells, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The present method can generate a high-quality immune cell preparation in a short period of 3 to 4 days with high efficiency. The present methods can be performed in a closed system for preparation of immune cells. In certain embodiments, the present method uses CliniMACS Prodigy® as the main culture system, and uses Dynabeads® to activate cells.

There is a TCR/CD3 complex on the surface of T cells. A first signal of cell activation is transmitted through CD3, thereby activating a variety of signal transduction pathways of T cells. Then, a second signal of T cell activation is generated by the interaction of CD28 binding molecule with the CD28 costimulatory molecule on the surface of T cells, thereby inducing proliferation and differentiation of T cells. All of the activation reagents Dynabeads®, TransAct™ and OKT3 activate T cells through this activation principle.

Compared to the standard CliniMACS Prodigy® method, the present method can greatly increase the sample amount/volume. A sufficient amount of cell products with a high proportion of Tnaive and Tcm can be obtained within only 3 to 4 days.

Samples that can be treated include, but are not limited to, fresh peripheral blood, fresh apheresis collections, cryopreserved apheresis collections, PBMC collections, T cells, NK cells, etc.

The media that can be used in the process system include, but are not limited to, media containing serum, low-serum media and serum-free media.

The sample pretreatment process may use equipment including, but not limited to, Sepax 2, Sepax C-pro, Sefia, Lovo, CS 5+, CS Elite and centrifugal equipment, etc.

The volume of the sample that can be treated may range from 5 to 500 ml. The centrifugal force for washing the sample may range from 100 to 1,000 g. The centrifugation time for washing the sample may range from 100 to 600 seconds. The dilution ratio for the sample may range from 0 (no dilution) to 5 times. The number of washing cycles may range from 1 to 5. The output volume after washing may range from to 400 ml.

The fully automatic, closed culture process may be a short cycle of 3 to 4 days. The initial loading of monocytes to the system may range from 1×10⁵ to 50×10⁹ cells. Cell sorting includes, but is not limited to, positive sorting based on, e.g., CD4⁺, CD8⁺, CD62L⁺, CD3⁺, CD56⁺ and combinations thereof, and negative sorting based on, e.g., CD14⁺, CD19⁺, CD269⁺ and combinations thereof. The dosage ratio may range from 50 to 5,000×10⁶ labelled cells/ml.

Cell activation density may range from 0.1×10⁶/ml to 20×10⁶/ml.

Activation reagents include, but are not limited to, TransAct™, microbeads as described herein (e.g., Dynabeads®), anti-CD3 antibodies (e.g., the OKT3 antibody) and other activation reagents. The dosage range of TransAct™ includes, but is not limited to, 0.1 ml to 30 ml. The microbead to cell ratio may range from 0.1 to 10. The dosage range of the OKT3 antibody includes, but is not limited to, 10 ng/ml to 50 mg/ml.

Activation time may range from about 4 hours to about 96 hours. The amount of activated cells may range from 1×10⁵ to 20×10⁹.

Genetical modification of the cells may use a non-viral transfection system (such as electroporation, e.g., a Neon transfection system, Maxcyte etc.) or a viral transduction system (such as a lentiviral system, an adenovirus system, and an adeno-associated virus vector, etc.

For transduction, the MOI may range from 0 to 1,000. The titer of the viral vector may range from about 1×10⁵ TU/ml to about 10×10¹⁹ TU/ml. The cell density may range from 0.01×10⁶/ml to 20×10⁶/ml.

The sample output volume during cell washing and concentration may range from 50 to 200 ml.

The cell washing and concentration equipment includes, but is not limited to, Sepax 2, Sepax C-pro, Sefia, Lovo, CS 5+, CS Elite, Prodigy, etc. The volume of the sample that can be treated includes but is not limited to 5 to 500 ml. The centrifugal force for washing the sample includes but is not limited to 100 to 1,000 g. The centrifugation time for washing the sample includes but is not limited to 100 to 600 seconds. The dilution ratio of the sample includes but is not limited to 0 to 5 times. The number of washing cycles includes but is not limited to 1 to 5. The output volume after concentration includes but is not limited to 5 to 400 ml.

The cell dispensing equipment includes but is not limited to Sepax 2, Sepax C-pro, Sefia, Prodigy dispensing equipment, etc. The volume of the sample that can be treated includes but is not limited to 5 ml to 500 ml. The output volume of dispensing includes but is not limited to 5 ml to 400 ml.

The comparison of the technical effects between present method and the standard CliniMACS Prodigy® method is shown in Table A below.

TABLE A
Comparison of the Immune Cell Preparation Method
of the Present Invention and the Method Officially
Recommended by CliniMACS Prodigy

| | An embodiment | Standard |
|---|---|---|
| | of the present | CliniMACS |
| | method | Prodigy ® method |
| Cryopreserved apheresis | 92.4% | N/A |
| collection pretreatment | | |
| (Recovery rate) | | |
| Sorting recovery rate | 80.2% | 35.7% |
| Sorting reagent cost | ~$1550 | ~$7800 |
| Proportion of Tnaive after | 40.5% | 14.0% |
| sorting | | |
| Cell number at inoculation | 400 × 10⁶ | 200 × 10⁶ |
| Proportion of activated cells | 95.1% | 70.4% |
| Positive rate | 68.4% | 31.2% |
| Number of culturing days | 4 days | 10 to 14 days |
| Cell number at final harvest | 4170 × 10⁶ | 3950 × 10⁶ |
| Total cell expansion times | 10.4 | 19.7 |
| Number of positive cells at the | 2852.3 × 10⁶ | 1232.4 × 10⁶ |
| final harvest | | |
| Tnaive/Tcm ratio at harvest | 39.4%/35.2% | 6.7%/18.5% |
| Cell concentration and | 95% | N/A |
| dispensing recovery rate | | |

Compared with the standard CliniMACS Prodigy® method, the present method may include an addition pretreatment step. The present method may include an improved sorting process to significantly increase the sorting recovery rate (from 35.7% to 80.2%) and the proportion of Tnaive after sorting (from 31.2% to 68.4%). This also reduces the costs of sorting reagents. The present method may improve the cell activation step. Even when the number of cells to be treated is doubled (from 200×10⁶ to 400×10⁶), the proportion of activated cells (from 70.4% to 95.1%) and the positive rate (from 31.2% to 68.4%) can still be significantly increased. Importantly, the time period for the present method is shortened from 10 to 14 days (for standard CliniMACS Prodigy® method) to 3-4 days. The number of positive cells and the Tnaive/Tcm ratio at harvest are significantly greater than those obtained by the standard CliniMACS Prodigy® method.

Table B compares each step of the present invention with the standard CliniMACS Prodigy® method.

TABLE B
Comparison of an embodiment of the present method with CliniMACS Prodigy

| | | Standard CliniMACS |
|---|---|---|
| | An embodiment of the present method | Prodigy ® method |
| Cryopreserved | Sepax C-pro is used for automatic washing | N/A |
| apheresis | to remove cryopreserved solution and | |
| collection | restore cells to good condition. | |
| pretreatment | | |
| Sorting | Pretreated cells and diluted sorting reagents | The sorting method of |
| | are used for sorting so that the cells are | the standard |
| | restored to their state after pretreatment, | CliniMACS Prodigy ® |
| | which will increase the sorting efficiency. | method is used, using |
| | | untreated sorting |
| | | reagents. |
| Activation | Microbeads (e.g., Dynabeads ®) are used for | TransAct ™ is used. |
| | activation. The activation effect is | |
| | significantly better than TransAct ™. | |
| | (Flow cytometry shows that microbeads | |
| | (e.g., Dynabeads ®) result in (i) | |
| | significantly higher proportion of activated | |
| | cells, (ii) better cell expansion, (iii) | |
| | significantly higher positive rate of cells, | |
| | (iv) significantly higher proportion of | |
| | Tnaive and Tcm cells, compared to those | |
| | when TransAct ™ is used for activation. | |
| | In addition, the costs of using Dynabeads are | |
| | lower than those of using TransAct ™. | |
| Virus | The transduction method of the standard | The transduction |
| transduction | CliniMACS Prodigy method is used. | method of the standard |
| | | CliniMACS Prodigy ® |
| | | method is used. |
| Virus removal | The virus removal method of the standard | The virus removal |
| | CliniMACS Prodigy method is used. | method of the standard |
| | | CliniMACS Prodigy ® |
| | | method is used. |
| Time period | 3-4 days | 14 days |
| Cell | Sepax C-pro is used for automatic | N/A |
| concentration | concentration and dispensing to ensure | |
| and | sterility and consistency, and to avoid lot- | |
| dispensing | to-lot variability due to manual operation. | |

In certain embodiments, the microbeads (e.g., Dynabeads®) are monodisperse/homogeneous, superparamagnetic and polymeric microspheres comprising γFe₂O₃ and Fe₃O₄ magnetic materials. The microbeads are coated with a layer of polymeric material, which acts as a carrier for adsorbing or binding antibodies specific for CD3 and/or CD28 cell surface molecules.

The main advantages of the present methods are as follows. The present method can greatly increase the sample treatment volume. A sufficient amount of cell products with a high proportion of Tnaive and Tcm can be obtained within only 3 to 4 days. The present method uses microbeads (e.g., Dynabeads®) to activate cells. Compared with the activation reagent TransAct™, the generated cell number and positive rate are higher. The present method can use a fully automatic, closed system. The preparation efficiency of immune cells is high, with good quality control.

The present disclosure provides a high-efficiency, integrated immune cell preparation method. Compared with the standard CliniMACS Prodigy® method, the present method includes an additional pretreatment of apheresis collections, optimized cell sorting, optimized cell activation, and an additional cell concentration and dispensing step.

Example 1 describes the pretreatment of a sample. Example 2 describes the further processing of the pretreated sample by using an embodiment of the present method. Example 3 describes the further processing of the pretreated sample by using the method recommended by CliniMACS Prodigy®. In Example 4, an embodiment of the present method was used, except that the cell sorting was performed based the method recommended by CliniMACS Prodigy®. In Example 5, an embodiment of the present method was used, except that the cell activation was performed by the method recommended by CliniMACS Prodigy®.

The present disclosure is further detailed below in conjunction with specific embodiments. It should be understood that these embodiments are only used to illustrate the present invention but not to limit the scope of the present invention. The experimental methods that do not indicate specific conditions in the following embodiments usually follow the conventional conditions or the conditions recommended by the manufacturer. Unless otherwise stated, percentages and parts are calculated by weight.

### Example 1 Sample Pretreatment

Sample pretreatment was carried out as follows:

1) 10 to 100 ml of a cryopreserved apheresis collection was thawed. The total number of cells was determined. 2/5 of the cell suspension was divided into two parts for use in Example 3 and Example 6.

2) Cells were washed using the Sepax Pro “Culture Wash” program. The parameters were set as follows: “Initial volume”=40 ml; “Final volume”=60 ml; “Dilution ratio”=2; “number of washing cycles”=2; “Intermediate volume”=20 ml; “g-Force”=300 g; “Dilution rate”=60 ml/min; “Settling time”=360 seconds. The washing solution was HSA solution, with the final concentration of HSA being 0.1% to 30%.

3) After washing, the new sample bag was removed, and the total number of cells was measured.

4) The cells were divided into three groups to be used in Example 2, Example 4 and Example 5, respectively.

### Results

The sample pretreatment by Sepax Pro resulted in good recovery rates for both monocytes and lymphocytes. The specific results are shown in Table 1.

TABLE 1
Recovery Rate of Monocytes and Lymphocytes after Pretreatment

| | Monocyte | Lymphocytes |
|---|---|---|
| Apheresis collection | 3000 × 10⁶ | 2560 × 10⁶ |
| After washing the apheresis collection | 2880 × 10⁶ | 2493 × 10⁶ |
| Recovery rate | 96.0% | 97.4% |

### Example 2 Short-Cycle Cultivation and Harvest Experiment

The pretreated sample from Example 1 was subjected to a fully automatic, closed culture system for a short period of 3 to 4 days. The specific method was as follows:

1) Cell preparation. A pretreated sample from Example 1 was connected to a Prodigy® line.
2) Cell sorting. An apheresis collection was positively sorted with anti-CD4 and anti-CD8 antibodies. Sorting reagents were diluted with PBS-EDTA containing 0.1% to 10% HAS. The dilution method was as follows. 7.5 ml antibodies were dispensed into 1.5 ml/bottle, then 3.5 ml PBS-EDTA containing 0.1% to 10% HSA was added to prepare a 5-ml diluted sorting system. During sorting, positive sorting was performed using anti-CD4 and anti-CD8 antibodies.
3) Cell activation. The sorted cells were activated with coated microbeads (e.g., Dynabeads®), and the cell activation state was analyzed with flow cytometry 24 hours after activation. The specific activation method was as follows. 10×10⁶ to 1000×10⁶ cells (e.g., 400×10⁶ cells) were activated with coated microbeads (e.g., Dynabeads®), where the bead to cell ratio ranged from 0.5 to 5 (e.g., about 1:1). The activation density was 0.5 to 10×10⁶/ml.
4) Virus transduction. After 1-2 days of cell activation, lentiviruses were added at the MOI of 1 to 10 (e.g., 3).
5) Virus removal. After 1-2 days of incubation, viruses were removed using Prodigy®.
6) Cell culture and harvest. The cells were cultured for additional 1 to 2 days, and then harvested with Prodigy®. The harvest volume was 50 to 200 ml.
7) Cell concentration. Cells were concentrated using Sepax Pro, with the following parameters: “Initial volume”=100 ml; “Final volume”=21 ml; “Dilution ratio”=0; “number of washing cycles”=1; “Intermediate volume”=20 ml; “g-Force”=300 g; “Dilution rate”=60 ml/min; “Settling time”=360 s. After the concentration step, the sample bag was remove and the concentration and volume of the cell suspension were determined.
8) Cell dispensing. The cells were dispensed using Sepax Pro, where the dispensing volume was 10 to 80 ml. After the dispensing step, the sample bag was removed, and the volume and cell density were determined.

### Results

The above method achieved cell sorting, activation, transduction and expansion within 3 to 4 days, and produced the desired cell numbers. The recovery rate of cell sorting is shown in Table 2.

TABLE 2
Recovery Rate of Cell Sorting with Prodigy

| CD4⁺ + CD8⁺T cells in an apheresis collection | 561 × 10⁶ |
|---|---|
| CD4⁺ + CD8⁺T cells after sorting | 450 × 10⁶ |
| CD4⁺ + CD8⁺T cell sorting recovery rate | 80.2% |

When Dynabeads® were used for activation, 95.1% of the cells were in an activated state after 24 hours. The cells continued to expand when cultured further. The specific results are shown in Table 3.

TABLE 3
Cell Activation Status after Activation

| Day 0: cell number | 400 × 10⁶ |
|---|---|
| Day 1: cell activation percentage | 95.1% |
| Day 3: cell number | 1200 × 10⁶ |
| Day 3: positive rate | 27.0% |
| Day 4: cell number | 4170 × 10⁶ |
| Day 4 positive rate | 54.2% |

T cells were cultured in a short period of 3 to 4 days in a fully closed automatic manner. The cell subsets were assayed in the apheresis collection, after sorting on Day 0, Day 1, Day 3, and Day 4. It was found that this method could produce a high proportion of Tnaive and Tcm cells. The specific results are shown in Table 4.

TABLE 4
Cell Subset Information After Activation with Dynabeads ®

| | Tnaive | Tcm |
|---|---|---|
| Apheresis collection | 45% | 35% |
| Day 0 (after sorting) | 43% | 31% |
| Day 1 | 42% | 30% |
| Day 3 | 39% | 34% |
| Day 4 | 39.4% | 35.2% |

Sepax Pro was used to concentrate the cell sample, which ensured the output volume and a good recovery of monocytes and lymphocytes. The specific results are shown in Table 5 and Table 6 below. The results show that the recovery rate of monocytes and lymphocytes is higher than 98%.

TABLE 5
Cell Number and Recovery Rate Before and After Concentration

| | Monocyte | Lymphocytes |
|---|---|---|
| Cell number before concentration | 4000 × 10⁶ | 3950 × 10⁶ |
| Cell number after concentration | 3920 × 10⁶ | 3886.8 × 10⁶ |
| Recovery rate | 98% | 98.4% |

TABLE 6
Cell Suspension Volume and Density
Before and After Concentration

| | Set value/ | |
|---|---|---|
| | theoretical | Actual |
| | value | volume |
| Cell suspension volume after concentration | 20 ml | 20.1 ml |
| Cell suspension density after concentration | 10 × 10⁶/ml | 9.9 × 10⁶/ml |

Sepax Pro was used to dispense the cell sample, which ensured the output volume and the consistency of the cell density during dispensing. The specific results are shown in Table 7 below. The results show that the sample volume and concentration after dispensing are substantially consistent with the theoretical values.

TABLE 7
Sample Volume and Concentration After Dispensing

| | Set value/ | |
|---|---|---|
| | theoretical | Actual |
| | value | volume |
| Volume of a first dispensed bag of sample | 20 ml | 20.1 ml |
| Concentration of a first dispensed bag of | 10 × 10⁶/ml | 10.1 × 10⁶/ml |
| sample | | |
| Volume of a second dispensed bag of | 40 ml | 40.5 ml |
| sample | | |
| Concentration of a second dispensed bag | 10 × 10⁶/ml | 9.9 × 10⁶/ml |
| of sample | | |
| Volume of a third dispensed bag of sample | 200 ml | 202 ml |
| Concentration of a third dispensed bag of | 10 × 10⁶/ml | 9.9 × 10⁶/ml |
| sample | | |

### Example 3 Standard CliniMACS Prodigy® Method

The standard CliniMACS Prodigy® method was carried out without the pretreatment described in Example 1, as follows:

1) Cell preparation: the sample was connected to a Prodigy® line.

2) Cell sorting: an apheresis collection was positively sorted with an anti-CD4 antibody and an anti-CD8 antibody. The sorting reagents were used without dilution.

3) Cell activation: the sorted cells were activated with TransAct™. The cell activation status was analyzed with flow cytometry 24 hours after activation. Specifically, 10×10⁶ to 1,000×10⁶ cells (e.g., 200×10⁶ cells) were activated with TransAct™.

4) Virus transduction: after culturing for 1 to 2 days, the required volume of lentiviruses was added to achieve MOI 1 to 10 (e.g., 3).

5) Virus removal: after 1 to 2 days of incubation, viruses were removed using Prodigy®.

6) Cell culture and harvest: cells were cultured for 14 days, and then harvested using Prodigy®. The harvest volume was 50 to 200 ml.

The experimental results are shown in Table 8.

TABLE 8
Recovery Rate of Cell Sorting with Prodigy ®

| CD4⁺ + CD8⁺T cells in an apheresis collection | 561 × 10⁶ |
|---|---|
| CD4⁺ + CD8⁺T cells after sorting | 200 × 10⁶ |
| CD4⁺ + CD8⁺T cell sorting recovery rate | 35.7% |

When TransAct™ was used for activation, after 24 hours of culture, 70.4% of the cells were activated. The cells continued to expand when cultured further. The specific results are shown in Table 9.

TABLE 9
Cell Activation Situation after Activation

| Day 0: cell number | 200 × 10⁶ |
|---|---|
| Day 1: cell activation percentage | 70.4% |
| Day 3: cell number | 112 × 10⁶ |
| Day 3: positive rate | 27.0% |
| Day 14: cell number | 3950 × 10⁶ |
| Day 14: positive rate | 31.2% |

Culturing was conducted by the standard CliniMACS Prodigy® method. The cell subsets were assayed in the apheresis collection, after sorting on Day 0, Day 1, Day 3, and Day 4. The specific results are shown in Table 10.

TABLE 10
Cell Subset Information after Activation with TransAct ™

| | Tnaive | Tcm |
|---|---|---|
| Apheresis collection | 45% | 35% |
| Day 0 (after sorting) | 43% | 31% |
| Day 1 | 42% | 27% |
| Day 3 | 25% | 20% |
| Day 14 | 6.7% | 18.5% |

Compared to an embodiment of the present method described in Example 2, the method of this Example contained no cell pretreatment step, and used cell sorting and cell activation methods of the standard CliniMACS Prodigy® method. The results show that the method of this Example resulted in lower cell sorting recovery rate after cell sorting, lower cell activation percentage, significantly lower final positive rate, significantly lower cell expansion fold, significantly lower proportions of Tnaive and Tcm cells, compared to an embodiment of the present method described in Example 2. In addition, the costs of cell sorting and activation in Example 2 were significantly lower than that by the standard CliniMACS Prodigy® method. In terms of the culture time period, Example 2 greatly shortened the culture time than that by the standard CliniMACS Prodigy® method.

### Example 4

A culture experiment was performed on a sample pretreated in Example 1. The specific method is as follows:

1) Cell preparation: a pretreated sample from Example 1 was connect to a Prodigy® line.

2) Cell sorting: an apheresis collection was positively sorted with an anti-CD4 antibody and an anti-CD8 antibody. The sorting reagents were used without dilution.

3) Cell activation: The sorted cells were activated with coated microbeads (e.g., Dynabeads®), and the cell activation state was analyzed with flow cytometry 24 hours after activation. The specific activation method was as follows. 10×10⁶ to 1000×10⁶ cells (e.g., 400×10⁶ cells) were activated with coated microbeads (e.g., Dynabeads®), where the bead to cell ratio ranged from 0.5 to 5 (e.g., about 1:1). The activation density was 0.5 to 10×10⁶/ml.

4) Virus transduction. After 1-2 days of cell activation, lentiviruses were added at the MOI of 1 to 10 (e.g., 3).

5) Virus removal: After 1-2 days of incubation, viruses were removed using Prodigy®.

6) Cell culture and harvest: The cells were cultured for 4 days, and then harvested with Prodigy®. The harvest volume was 50 to 200 ml.

The experimental results are shown in Table 11.

TABLE 11
Recovery Rate of Cell Sorting with Prodigy ®

| CD4⁺ + CD8⁺ T cells in an apheresis collection | 561 × 10⁶ |
|---|---|
| CD4⁺ + CD8⁺ T cells after sorting | 439 × 10⁶ |
| CD4⁺ + CD8⁺ T cell sorting recovery rate | 78.3% |

When Dynabeads® was used for activation, after 24 hours of culture, 89.2% of the cells were activated. The cells continued to expand when cultured further. The specific results are shown in Table 12.

TABLE 12
Cell Activation Situation after Activation

| Day 0: cell number | 400 × 10⁶ |
|---|---|
| Day 1: cell activation percentage | 89.2% |
| Day 3: cell number | 950 × 10⁶ |
| Day 3: positive rate | 23.5% |
| Day 14: cell number | 3350 × 10⁶ |
| Day 4: positive rate | 49.3% |

Culturing was conducted by the standard CliniMACS Prodigy® method. The cell subsets were assayed in the apheresis collection, after sorting on Day 0, Day 1, Day 3, and Day 4. The specific results are shown in Table 13.

TABLE 13
Cell Subset Information After Activation with Dynabeads ®

| | Tnaive | Tcm |
|---|---|---|
| Apheresis collection | 45% | 35% |
| Day 0 (after sorting) | 40% | 32.2% |
| Day 1 | 42% | 36.2% |
| Day 3 | 38.2% | 33.2% |
| Day 4 | 35.4% | 30% |

Compared to an embodiment of the present method described in Example 2, the method of this Example used the cell sorting protocol of the standard CliniMACS Prodigy® method. The results show that the cell sorting recovery rate of this Example was not significantly different from that of Example 2. The subsequent cell activation, cell expansion, final positive rate, and Tnaive and Tcm at harvest were also not significantly different from those of Example 2. In the sorting step of Example 2, the dispensing and sorting of magnetic beads greatly reduced the costs and did not affect the subsequent state of the cells.

### Example 5

Culture experiment was performed on a sample from Example 1. The specific method was as follows:

1) Cell preparation: A pretreated sample from Example 1 was connected to a Prodigy® line.

2) Cell sorting: An apheresis collection was positively sorted with anti-CD4 and anti-CD8 antibodies. Sorting reagents were diluted with PBS-EDTA containing 0.1% to 10% HAS. The dilution method was as follows. 7.5 ml antibodies were dispensed into 1.5 ml/bottle, then 3.5 ml PBS-EDTA containing 0.1% to 10% HSA was added to prepare a 5-ml diluted sorting system. During sorting, positive sorting was performed using anti-CD4 and anti-CD8 antibodies.

3) Cell activation: the sorted cells were activated with TransAct™. The cell activation status was analyzed with flow cytometry 24 hours after activation. Specifically, 10×10⁶ to 1,000×10⁶ cells (e.g., 200×10⁶ cells) were activated with TransAct™.

4) Virus transduction. After 1-2 days of cell activation, lentiviruses were added at the MOI of 1 to 10 (e.g., 3).

5) Virus removal. After 1-2 days of incubation, viruses were removed using Prodigy®.

6) Cell culture and harvest. The cells were cultured for 4 days, and then harvested with Prodigy®. The harvest volume was 50 to 200 ml.

The experimental results are shown in Table 14.

TABLE 14
Recovery Rate of Cell Sorting in Example 5

| CD4⁺ + CD8⁺ T cells in an apheresis collection | 561 × 10⁶ |
|---|---|
| CD4⁺ + CD8⁺ T cells after sorting | 400 × 10⁶ |
| CD4⁺ + CD8⁺ T cell sorting recovery rate | 71.3% |

When TransAct™ was used for activation, after 24 hours of culture, 72.6% of the cells were activated. The cells continued to expand when cultured further. The specific results are shown in Table 15.

TABLE 15
Cell Activation Situation after Activation

| Day 0: cell number | 400 × 10⁶ |
|---|---|
| Day 1: cell activation percentage | 71.3% |
| Day 3: cell number | 351 × 10⁶ |
| Day 3: positive rate | 15.7% |
| Day 14: cell number | 1053 × 10⁶ |
| Day 4: positive rate | 39.5% |

Culturing was conducted by the standard CliniMACS Prodigy® method. The cell subsets were assayed in the apheresis collection, after sorting on Day 0, Day 1, Day 3, and Day 4. The specific results are shown in Table 16.

TABLE 16
Cell Subset Information after Activation

| | Tnaive | Tcm |
|---|---|---|
| Apheresis collection | 45% | 35% |
| Day 0 (after sorting) | 40.2% | 32.4% |
| Day 1 | 38.5% | 27.2% |
| Day 3 | 25.3% | 26.2% |
| Day 4 | 16.2% | 19.6% |

The difference between the method used in this Example and Example 2 is the cell activation method. The cell activation was using TransAct™ in this Example, while Dynabeads® was used in Example 2. In this Example, the cell activation ratio, cell expansion, final positive rate, and Tnaive and Tcm at harvest were significantly lower than those in Example 2.

### Example 6

The pretreated sample from Example 1 was subjected to a fully automatic, closed culture system for a short period of 3 to 4 days. The specific method was as follows:

1) Cell preparation. A pretreated sample from Example 1 was connected to a Prodigy® line.

2) Cell sorting. An apheresis collection was positively sorted with anti-CD4 and anti-CD8 antibodies. Sorting reagents were diluted with PBS-EDTA containing 0.1% to 10% HAS. The dilution method was as follows. 7.5 ml antibodies were dispensed into 1.5 ml/bottle, then 3.5 ml PBS-EDTA containing 0.1% to 10% HSA was added to prepare a 5-ml diluted sorting system. During sorting, positive sorting was performed using anti-CD4 and anti-CD8 antibodies.

3) Cell activation: The sorted cells were activated with coated microbeads (e.g., Dynabeads®), and the cell activation state was analyzed with flow cytometry 24 hours after activation. The specific activation method was as follows. 10×10⁶ to 1000×10⁶ cells (e.g., 317×10⁶ cells) were activated with coated microbeads (e.g., Dynabeads®), where the bead to cell ratio ranged from 0.5 to 5 (e.g., about 1:1). The activation density was 0.5 to 10×10⁶/ml.

4) Virus infection. After 1-2 days of cell activation, lentiviruses were added at the MOI of 1 to 10 (e.g., 3).

5) Virus removal. After 1-2 days of incubation, viruses were removed using Prodigy®.

6) Cell culture and harvest. The cells were cultured for additional 1 to 2 days, and then harvested with Prodigy®. The harvest volume was 50 to 200 ml.

The experimental results are shown in Table 17.

TABLE 17
Recovery Rate of Cell Sorting with Prodigy

| CD4⁺ + CD8⁺T cells in an apheresis collection | 561 × 10⁶ |
|---|---|
| CD4⁺ + CD8⁺T cells after sorting | 317 × 10⁶ |
| CD4⁺ + CD8⁺T cell sorting recovery rate | 56.5% |

When Dynabeads® was used for activation, after 24 hours of culture, 75.8% of the cells were activated. The cells continued to expand when cultured further. The specific results are shown in Table 18.

TABLE 18
Cell Activation Situation after Activation

| Day 0: cell number | 317 × 10⁶ |
|---|---|
| Day 1: cell activation percentage | 88.5% |
| Day 3: cell number | 698 × 10⁶ |
| Day 3: positive rate | 22.4% |
| Day 4: cell number | 2156 × 10⁶ |
| Day 4: positive rate | 48.2% |

Culturing was conducted a short period of 3 to 4 days in a fully closed automatic manner. The cell subsets were assayed in the apheresis collection, after sorting on Day 0, Day 1, Day 3, and Day 4. The specific results are shown in Table 19.

TABLE 19
Cell Subset After Activation with Dynabeads ®

| | Tnaive | Tcm |
|---|---|---|
| Apheresis collection | 45% | 35% |
| Day 0 (after sorting) | 41% | 31.5% |
| Day 1 | 40.5% | 35.2% |
| Day 3 | 37.3% | 33.9% |
| Day 4 | 33.6% | 29.4% |

The difference between the method used in this Example and in Example 2 was that the cells were not pretreated in this Example. The results show that the cell sorting recovery rate was significantly lower than that in Example 2. The cell activation ratio, final positive rate, Tnaive and Tcm at harvest were not much different. Due to the low recovery rate of cell sorting, the total number of cells at harvest was low during the culture period of 3 to 4 days.

### Example 7

This Example further compares the effects of Dynabeads® and TransAct™ on cell activation and subsequent expansion. The specific method was as follows:

1) Cell preparation. T cells were manually sort from 1,000×10⁶ apheresis collection. take 400×10⁶ cells were divided equally into two portions for the follow-up experiment.

2) Cell activation. 200×10⁶ cells were activated with Dynabeads®. The specific activation method was as follows. 10×10⁶ to 1,000×10⁶ cells (e.g., 200×10⁶ cells) were activated with Dynabeads®. The bead to cell ratio ranged from 0.5 to 5 (e.g., about 1:1). The activation density was 0.5 to 10×10⁶/ml (e.g., 2.86×10⁶/ml).

The other half of cells (200×10⁶ cells) were activated with TransAct™. The specific activation method was as follows. 0.2×10⁶ to 500×10⁶ cells (e.g., 200×10⁶ cells) were activated with a bottle of TransAct™. The activation density was 0.5 to 10×10⁶/ml (e.g., 2.86×10⁶/ml).

3) Virus transduction. After 1-2 days of cell culture (e.g., transduction was performed one day after activation), lentiviruses were added at the MOI of 1 to 10 (e.g., 2).

4) Virus removal: After 1 to 2 days of incubation (e.g., about 1 day), the sample was centrifuged to remove the lentiviral vectors.

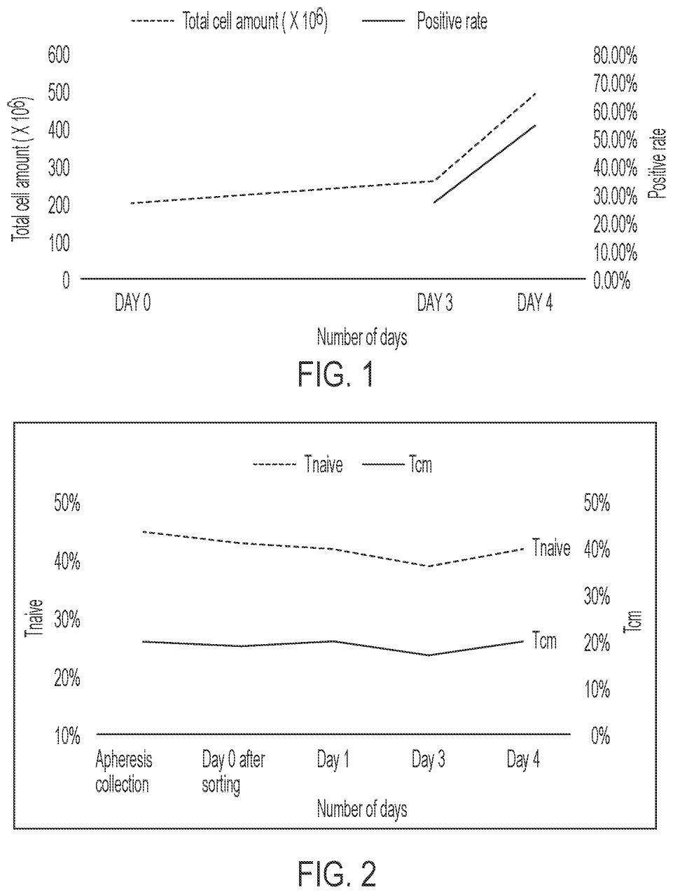

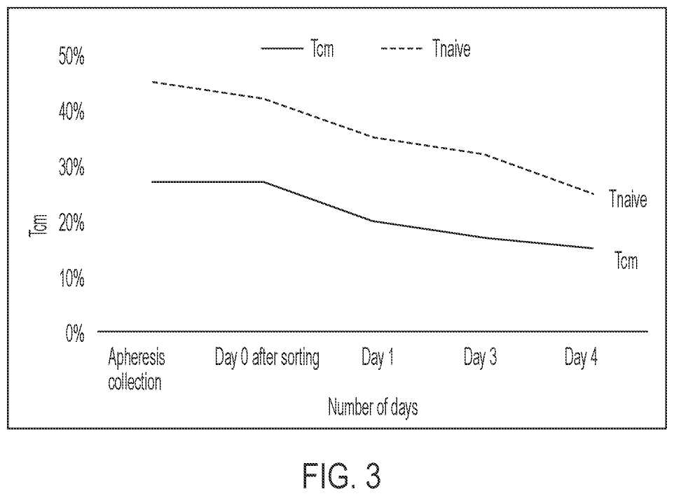

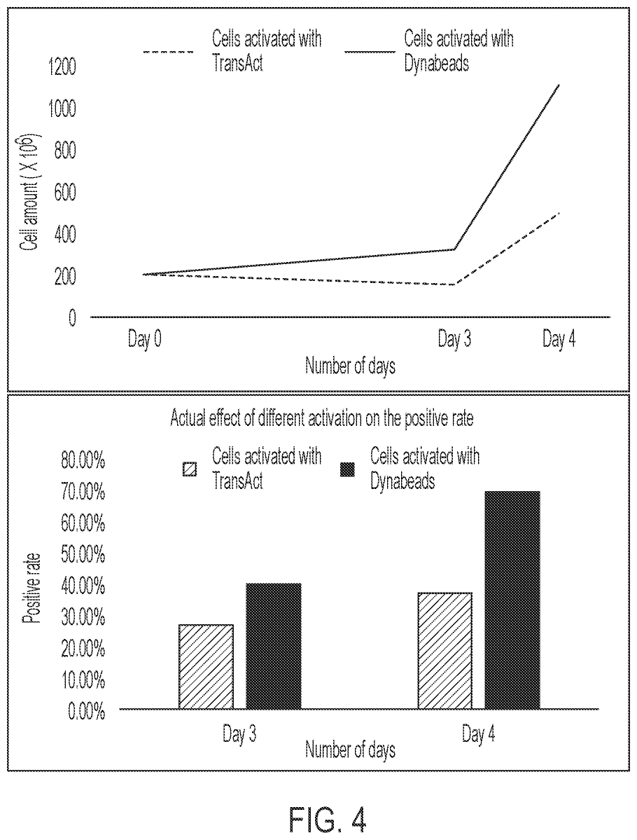

5) Cell culture and harvest. Cells were further cultured for 1 to 2 days. The cell subset information during culture is shown in Table 20, Table 21, FIG. 2 and FIG. 3. The cell number information during culture is shown in Table 22 and FIG. 4. The positive rate during culture is shown in Table 23 and FIG. 4.

TABLE 20
Percentage of Tniave Cells

| | Apheresis | | | | |
|---|---|---|---|---|---|
| | collection | 0 day after sorting | 1 day | 3 days | 4 days |
| TransAct ™ | 45% | 42% | 35% | 32% | 25% |
| Dynabeads ® | 45% | 42% | 41% | 38% | 41% |

TABLE 21
Percentage of Tcm Cells

| | Apheresis | | | | |
|---|---|---|---|---|---|
| | collection | 0 day after sorting | 1 day | 3 days | 4 days |
| TransAct ™ | 27% | 27% | 20% | 17% | 15% |
| Dynabeads ® | 27% | 27% | 29% | 25% | 27% |

TABLE 22
Cell number during Culture

| | Day 0 | Day 3 | Day 4 |
|---|---|---|---|
| TransAct ™ | 200 × 10⁶ | 173 × 10⁶ | 527 × 10⁶ |
| Dynabeads | 200 × 10⁶ | 320 × 10⁶ | 1168 × 10⁶ |

TABLE 23
Positive Rate during Culture

| | Day 3 | Day 4 |
|---|---|---|
| TransAct ™ | 27% | 35% |
| Dynabeads | 39% | 68% |

The scope of the present disclosure is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present disclosure and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present disclosure have been shown and described, it will be obvious to those skilled in the art that changes and modifications may be made without departing from the spirit and scope of the invention. The matter set forth in the foregoing description and accompanying drawings is offered by way of illustration only and not as a limitation.

1. A method for culturing genetically modified immune cells, the method comprising:
(a) providing a sample containing immune cells;
(b) optionally, washing the sample to obtain pretreated immune cells;
(c) sorting the pretreated immune cells to obtain enriched immune cells;
(d) activating the enriched immune cells with microbeads coated with activating agents to obtain activated immune cells;
(e) genetically modifying the activated immune cells to obtain genetically modified immune cells; and
(f) expanding the genetically modified immune cells.

2. The method of claim 1, wherein the microbeads have a diameter ranging from about 1 μm to about 10 μm.

3. The method of claim 2, wherein the microbeads have a diameter ranging from about 2 μm to about 8 μm.

4. The method of claim 3, wherein the microbeads have a diameter ranging from about 4 μm to about 5 μm.

5. The method of claim 1, wherein in step (d) the activating is performed with a microbead-to-cell ratio ranging from about 0.1 to about 10.

6. The method of claim 5, wherein in step (d) the activating is performed with a microbead-to-cell ratio ranging from about 0.5 to about 5.

7. The method of claim 5, wherein in step (d) the activating is performed with a microbead-to-cell ratio ranging from about 1 to about 2.

8. The method of claim 1, wherein the activating agents are selected from the group consisting of: antibodies or fragments thereof, cytokines, recombinant costimulatory molecules, small drug inhibitors, and combinations thereof.

9. The method of claim 1, wherein the activating agents are anti-CD3 and/or anti-CD28 antibodies or fragments thereof.

10. The method of claim 1, wherein in step (d) the activating is performed for about 16 hours to about 48 hours.

11. The method of claim 1, wherein step (d), step (e) and step (f) are performed in about 2 days to about 5 days.

12. The method of claim 11, wherein step (d), step (e) and step (f) are performed in about 3 days to about 4 days.

13. The method of claim 1, wherein all steps of the method are performed in about 2 days to about 5 days.

14. The method of claim 13, wherein all steps of the method are performed in about 3 days to about 4 days.

15. The method of claim 1, wherein steps (c)-(f) are performed in a closed and sterile system.

16. The method of claim 1, wherein all steps of the method are performed in a closed and sterile system.

17. The method of claim 1, wherein the immune cells are T cells or T cell subsets.

18. The method of claim 1, wherein in step (d) the activating is performed with an immune cell density ranging from about 0.5×10⁶ cells/ml to about 10×10⁶ cells/ml.

19. The method of claim 18, wherein in step (d) the activating is performed with an immune cell density ranging from about 2×10⁶ cells/ml to about 3×10⁶ cells/ml.

20. The method of claim 1, wherein in step (e) the genetically modifying is transducing or transfecting.

21. The method of claim 1, wherein in step (e) the genetically modifying comprises introducing into the activated immune cells a polynucleotide encoding a chimeric antigen receptor (CAR) or a T cell receptor (TCR).

22. The method of claim 1, wherein in step (e) the genetically modifying comprises transducing the activated immune cells with lentiviral vectors, gamma-retroviral vectors, alpha-retroviral vectors, or adenoviral vectors.

23. The method of claim 1, wherein in step (e) the genetically modifying comprises transducing the activated immune cells with lentiviral vectors.

24. The method of claim 1, wherein in step (b) the washing comprises using a human serum albumin (HSA) solution having a final HSA concentration of 0.1% to 30%.

25. The method of claim 1, wherein in step (b) the washing comprises using a human serum albumin (HSA) solution having a final HSA concentration of 0.1% to 10%.

26. The method of claim 1, wherein in step (b) the washing comprises centrifuging the sample using a centrifugal force ranging from about 100×g to about 1,000×g.

27. The method of claim 1, wherein in step (b) the washing comprises centrifuging the sample for about 100 seconds to about 600 seconds.

28. The method of claim 1, wherein in step (b) the washing comprises diluting the sample about 0 to about 5 folds.

29. The method of claim 1, wherein in step (b) the washing comprises performing the washing cycle for 1 to 5 times.

30. The method of claim 1, wherein step (b) has an output volume ranging from about 5 ml to about 400 ml.

31. The method of claim 1, wherein in step (c) the sorting comprises using anti-CD4 and/or anti-CD8 antibodies or fragments thereof.

32. The method of claim 1, wherein the sample is peripheral blood, immune cells, monocyte collections, or peripheral blood mononuclear cells (PBMCs).

33. Genetically modified immune cells prepared by the method of claim 1.

34. A cell preparation comprising the genetically modified immune cells of claim 33.

35. A pharmaceutical composition comprising the genetically modified immune cells of claim 33.
